(19) United States
(12) Patent Application Publication
(10) Pub. No.: US 2003/0125286 A1
(43) Pub. Date: Jul. 3, 2003

(54) Transcriptional silencer protein NRF

(75) Inventors: Hansjorg Hauser, Braunschweig (DE); Mahtab Nourbakhsh, Braunschweig (DE)

Correspondence Address: MARSHALL, GERSTEIN & BORUN, 6300 SEARS TOWER, 233 SOUTH WACKER, CHICAGO, IL, 60606-6357, US

(73) Assignee: Gesellschaft fuer Biotechnologischer Forschung mbH (GBF)

(21) Appl. No.: 10/244,787

(22) Filed: Sep. 16, 2002

Related U.S. Application Data
(63) Continuation of application No. 09/490,206, filed on Jan. 24, 2000, now abandoned.
Application No. 09/490,206 is a continuation of application No. PCT/EP98/04661, filed on Jul. 24, 1998.

(30) Foreign Application Priority Data
Jul. 24, 1997 (EP) 97112704.8

(51) Int. Cl. A61K 48/00; C12P 21/02; C12N 5/06; C07H 21/04; C07K 14/475

(52) U.S. Cl. 514/44; 435/69.1; 435/320.1; 435/325; 530/350; 536/23.5

(57) ABSTRACT

NRF is a novel inhibitory transcription factor binding to specific DNA sequences and silencing transcriptional activity of proximal DNA-binding activators, e.g. NF-_{K}B binding sites.

NRF is a modulator protein of NF-_{K}B family members controlling genes of significant biomedical importance such as those encoding inflammatory cytokines, MHC proteins, cell adhesion molecules, and viruses. Based on this it represents a molecular target in the development of novel anti-inflammatory therapies for a variety of pathologic disorders such as ischemia, hemorrhagic and septic shock, allograft rejection, bacterial meningitis, acute airway inflammation and the pulmonary complications induced by cardiopulmonary bypass. Furthermore, this might apply for certain cancers and other diseases.

NRF as a target for drugs (obtained e.g. by IITS) is protected. Agonists as well as antagonists of NRF are also protected. Further applications such as the use of NRF protein and its fusion proteins or NRF DNA sequence in sense or antisense orientation in gene therapy are also protected.

This is a continuation of International Application No. PCT/EP98/04661 filed Jul. 24, 1998 (International Publication No. WO 99/05269 dated Feb. 4, 1999). The respective disclosures of International Application No. PCT/EP98/04661 and International Publication No. WO 99/05269 are incorporated herein by reference.

The invention concerns the transcriptional silencer protein NRF which is a novel inhibitory transcription factor, and several related subject matters. The background of the invention is as follows.

NF-_{K}B/rel Protein

The family of NF-_{K}B/rel transcription factors regulates a variety of promoters through specific DNA-binding sites. NF-_{K}B/rel-binding sites act as weak constitutive enhancers. However, many promoters which contain NF-_{K}B/rel-binding sites do not show base level activity. This is explained by the existence of silencer elements. Further to the constitutive enhancing activity of the NF-_{K}B binding sites, many inducers like viruses, TNF-A or PMA induce a signalling cascade that increases the activity of the NE-_{K}B enhancers transiently. These inducers lead to a transient inactivation of IkB, the cytoplasmic inhibitor of certain NF-_{K}B members. This results in a nuclear translocation of the prototype NF-_{K}B (a heterodimer of p50 and p65) and the activation of the above mentioned target genes by binding and activation of transcription.

The nuclear factor NF-_{K}B rel family is involved in the regulation of a number of genes which contribute to physiological activities, like inflammation and cell growth. Inflammatory pathology and cancer are often associated with the disregulation of theses genes, raising the possibility that the initiation of multiple pathologic processes is due to NF-_{K}B/rel-mediated transactivation. Inhibitors of NF-_{K}B activation may therefore have broad applications as novel therapeutics in human diseases. Natural repression mechanisms might play an important part in the control of disregulated NF-_{K}B/rel activity.

The IFN-β Negative Regulatory Element (NRE)

An example of a repression mechanism was found in the control of mammalian Interferon-β (IFN-β) gene expression. IFN-β genes are absolutely silent but can be transcriptionally activated in nearly all differentiated type of cells by viruses or double-stranded RNA.

In this promoter several positive regulatory domains (PRDs) are assembled within less than loo base pairs. The PRDII, which represents a binding site for NF-_{K}B/rel proteins and PRDI to which members of the IRF-family can bind are responsible for a basal expression level (1, 9, 14, 18, 38). For full activity of the IFN-β promoter further PRD sequences are required.

A minimal virus response element (VRE) was identified. It contains PRDI, PRDII and an extended negative regulatory domain (NRD). The NRD was found to repress a basal transcriptional activity in the absence of inducers (10, 11). Within this NRD, an 11 base pair element acts as a negative regulatory element (NRE) of the PRDII sequence. Although this NRE is physically overlapping with PRDII, it can act as a position-independent silencer of PRDII. (24)

NREs in Other Promoters

Examination of NF-_{K}B/rel-binding sites containing promoters for the presence of NRE-related sequences and functions revealed several elements with a loose sequence relationship to the IFN-β NRE.

In the HIV-1 promoter, two sequences with homology to the IFN-β NRE were found in a region which was functionally defined as a region of negative regulation for HIV-1 transcription (3, 22). Saksela and Baltimore (1993) have described a negatively acting element (termed _{K}NE) immediately upstream of the NF-_{K}B-binding site in the Ig_{K} intronic enhancer. The core of this 27-bp _{K}NE sequence shows a high homology to the IFN-β NRE. A negative regulatory element was shown to exist in the IL-2 receptor α chain promoter (33) also exhibiting some sequence similarity to the IFN-β NRE. Homology to NRE in the HTLV-I promoter was also found. The region in which this sequence is located cooperates positively with the 21-bp enhancer upon Tax protein activation (34). The inducibility of these promoters involves the activation of NF-_{K}B/rel binding sequences.

The NRE-related sequences contained within the promoters of HIV-1 and HTL V-1 and the IL-2R-α gene constitute functionally related silencer elements which repress the constitutive enhancing activity of NF-_{K}B/rel-binding sites from these promoters. Thus, NREs represent a new class of transcriptional repressor sequences with a silencing activity on the constitutive activity of NF-_{K}B/rel binding sites.

All NREs show similar properties with respect to binding of proteins from nuclear extracts, however, with distinguishable affinities. The distinct affinities reflect the silencing capacity of the NREs. The NRE-mediated silencing effect is relieved by enhancer-specific inducers like viruses, TAX or PMA. Despite this homology, the NF kB/rel DNA-binding sites from HIV-1-LTR and IFN-β promoter exhibit significant differences. These differences are based on the sequence specificities of the NF-_{K}B/rel-binding sites, but not on the sequences of the NREs.

Common Features of NRES:

The common features exerted by the presently known five NREs are: sequence homology, short length (11-13 bp), distance and position-independent action, specific interaction with NF-_{K}B/rel-binding sequences and indistinguishable binding patterns of nuclear factors. A considerable number of other NREs in various genes is expected to exist.

For example, sequence comparisons show NRE homology sequences within the promoters of the cell adhesion molecules ELAM-1 and CAM-1. A negatively acting element (termed kNE) with homology to the NRE core sequence was described immediately upstream of the NF-_{K}B-binding site in the Igk intronic enhancer (27).

Target Sequence Specificity of NRE-function:

The NREs do not act on the basal transcription machinery. (24) Up to now, only the NRE silencing of NF-_{K}B/rel enhancers has been found. However, other activating sequences may also be silenced by the NREs. This is supported by the identification of a silencer from the gastrin promoter (39) which is highly homologous to the described NREs. The gastrin promoter does not contain NF-_{K}B/rel or related binding sites, assuming that other enhancer(s) interact with the gastrin silencer (37).

Proteins Binding to NREs:

Sequence and functional homology of the NREs from different sources suggest that the binding factors to these NREs are distinct or identical.

In EMSA the IFN-β NRE (N) sequence is retarded to give two major complexes (bands), the faster one having a higher binding affinity than the slower migrating complex (24). All functional NRE sequences are retarded in exactly the same manner, giving rise to the two complexes. The ability to form indistinguishable complexes suggests that the factors binding to these oligonucleotides are identical. This was further confirmed by cross-competition experiments. All NREs compete with each other in the same way as by themselves. However, competition data also demonstrate that affinity within the different complexes differs. The highest affinity is exhibited by IFN-β NRE, followed by the IL-2 Rec α NRE. NREs from both HIV-1 and HTLV-I show a clearly lower affinity. The affinities of NREs to nuclear proteins roughly reflect their silencing capacity to the NF-_{K}B/rel enhancer.

UV-crosslinking data suggested that the proteins would have molecular weight(s) of about 100 KDa (24). The currently published experiments do not allow a determination of the number of factors that are involved in the NRE specific silencing function(s).

Known NF-_{K}B-repressors:

Recently, a nuclear NF-_{K}B/rel inhibitor was described. This factor inhibits DNA-binding of p50/p65 heterodimers in Adenovirus transformed cells (21). Obviously, this repressor acts by suppressing the induced NF-_{K}B-enhancer activity and is therefore distinct to the factor(s) which repress the constitutive NF-_{K}B-enhancer activity.

A Drosophila 43 KDa HMG1 protein called DSP1 (dorsal switch protein) was described. This protein inhibits the Dorsal enhancer by binding to a proximally located sequence (17). Furthermore, DSP-1 represses NF-_{K}B/rel-site mediated enhancement in mammalian cells. The human homologue to DSP-1 which was believed to act as the IFN-β specific silencing protein, has not yet been described.

The viral transactivator Tax of HTLV-1 is able to restore the activity of the HIV-1 NF-_{K}B enhancer silenced by any NRE. Similar to this observation, Salvetti et al. (29) described the repression of an NF-_{K}B/rel binding site in the human vimentin promoter by a negative element which would be relieved by Tax expression. Tax transactivates several promoters through the induction of NF-_{K}B/rel proteins by nuclear translocation of cytoplasmatic dimers and de novo synthesis of c-rel (16). It has been shown that Tax is able to induce nuclear translocation of NF-_{K}B/rel proteins retained in the cytoplasm through interaction with p 105 or p 100 (19, 23). Induction of NF-_{K}B/rel enhancing activity by the Tax protein would result in masking the inhibitory effect exerted by the NREs. The unresponsiveness of the IFN-β and HTLV-1 NF-_{K}B enhancers to Tax expression and thus the unaltered repression by NREs may be due to the sequence differences of the investigated enhancers. Such differences in binding and transactivation of the known NF-_{K}B/rel dimers are well documented (20).

Release from Repression:

The repressive effect of the IFN-β NRE on PRDII cannot be eliminated by viral infection although this leads to an induction of NF-_{K}B binding activity (24). The inducible derepression of PRDII is dependent on the interaction with PRDI, a binding site for IRF-proteins. Similarly, induced NF-_{K}B/rel activity due to viral infection is not sufficient to activate the HTLV-1 enhancer. Depending on the NF-_{K}B enhancer element and the inducing agent, derepression requires an additional activator. The concerted action of coactivators or the induction of a particular set of NF-_{K}B/rel binding proteins are sufficient for releasing the repression.

Virus induction does not affect the negative activity of the NRE on isolated PRDII. However, a 28 base pair fragment containing PRDI, PRDII and NRE functions as a minimal VRE indicating that it is the cooperative effect of PRDI and PRDII which is responsible for overcoming the NRE function after virus infection. These properties together with electromobility shifts and DNA-crosslinking data indicate that the proteins being responsible for the silencing effect are still bound to the NRE after transcriptional induction by virus infection. It was speculated that the silencing effect of NRE-binding factor(s) might be due to a masking of the NF-_{K}B/rel activator or to ‘locking’ of the basal transcriptional complex for NF-_{K}B/rel activation. It was further speculated that replacement, post-transcriptional or steric alterations of the factors bound to the PRDs could eliminate the negative activity of the silencer protein(s) (24).

Apart from the activation mechanism, NF-_{K}B sequences exhibit a basal constitutive activity. Most probably, this background activity is maintained by the binding of NF-_{K}B/rel proteins which are not cytoplasmatically retained by I-_{K}B, e. g. p50 dimers. This basal activity is repressed in a number of NF-_{K}B promoters including those regulating IFN-β, IL-2 receptors-α chain.

According to one embodiment the invention concerns a ssDNA

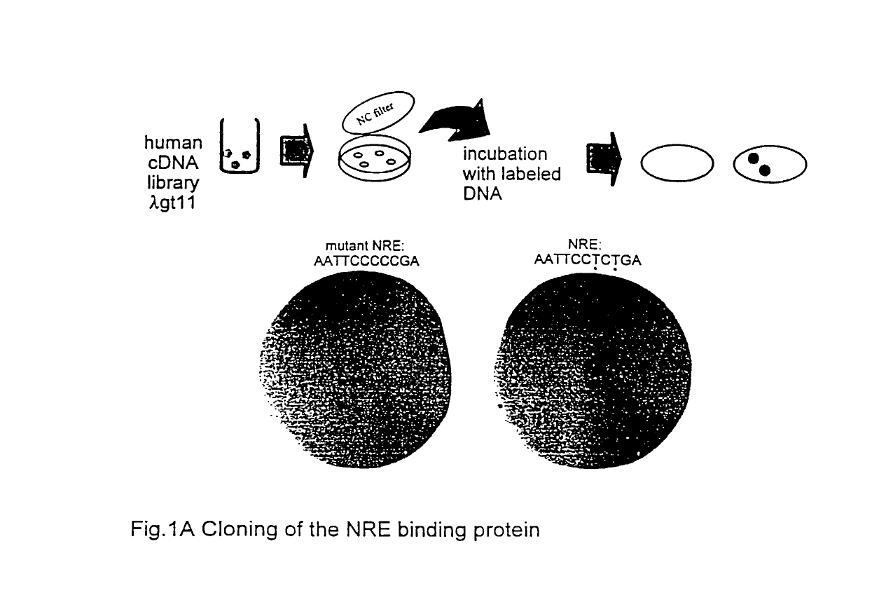

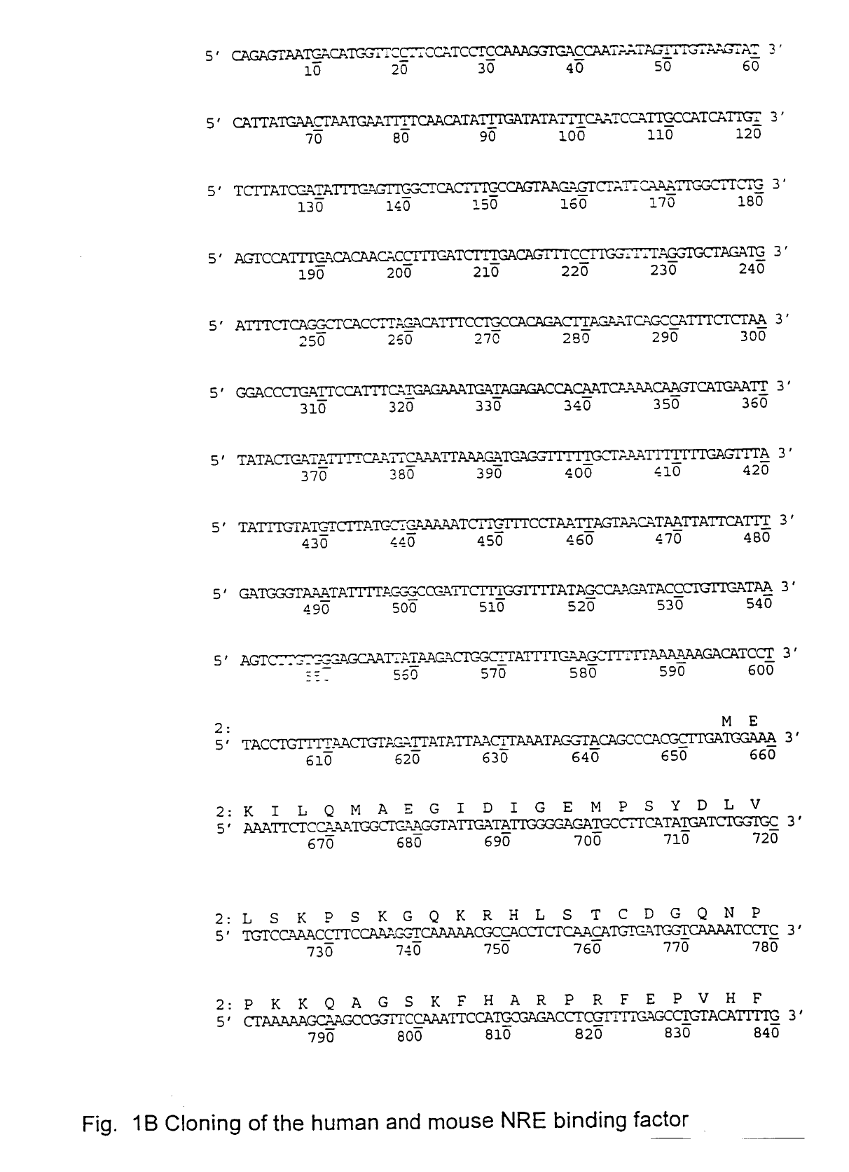

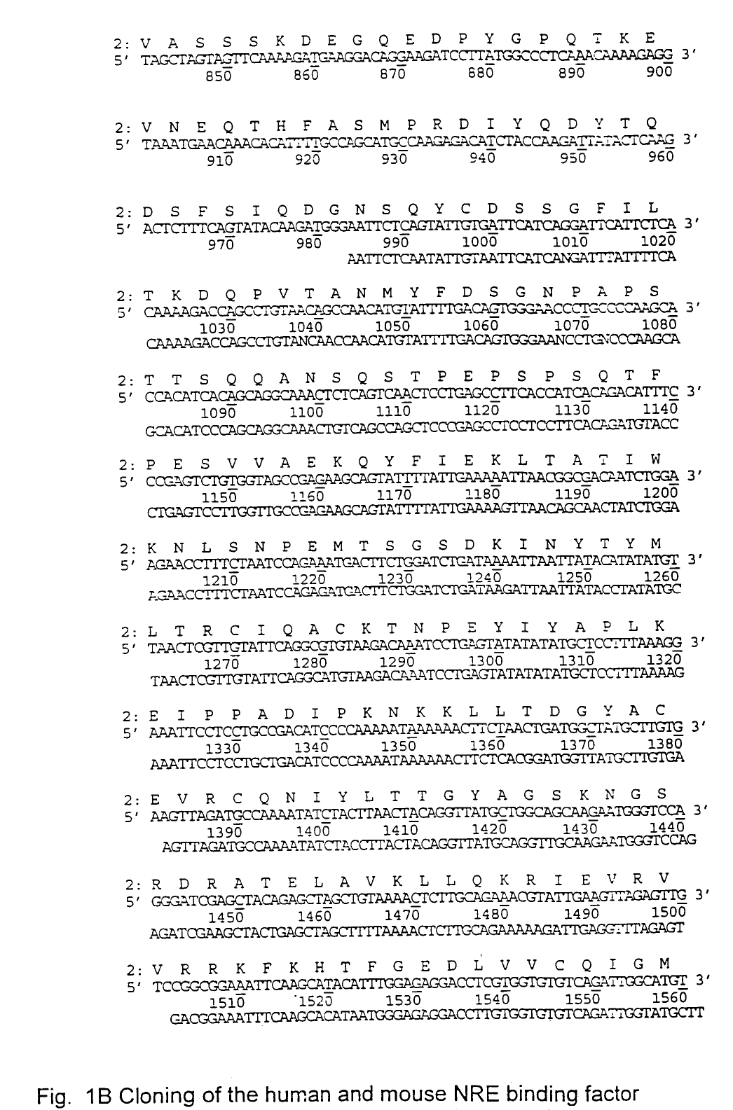

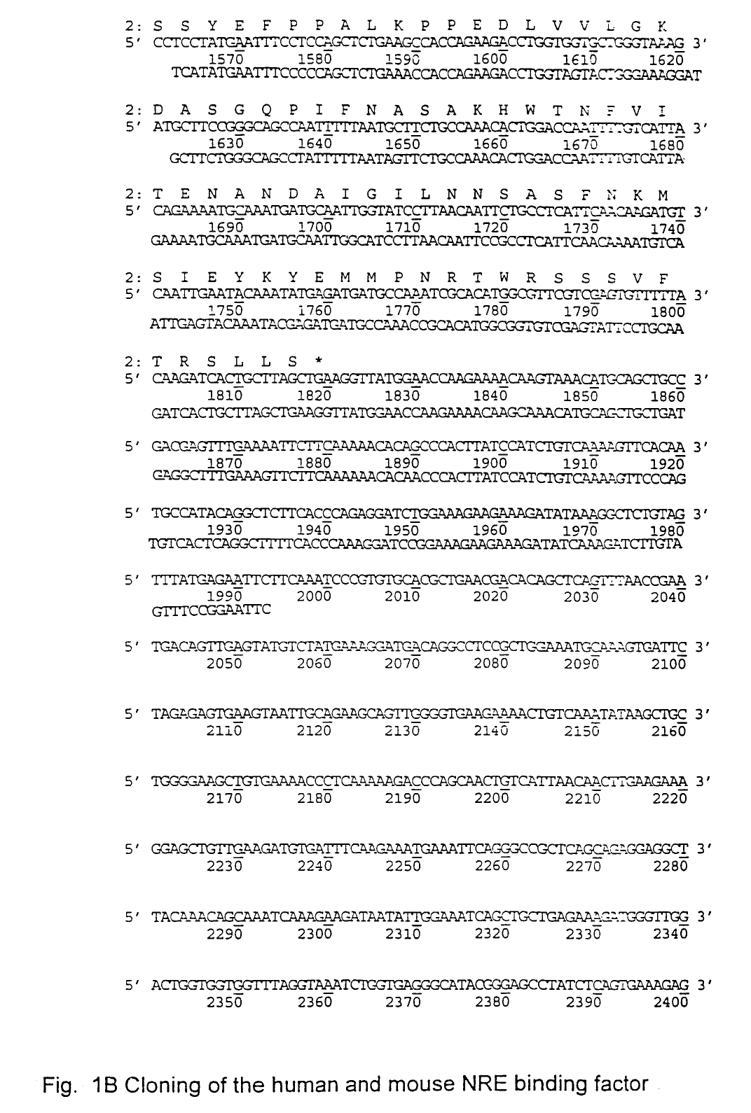

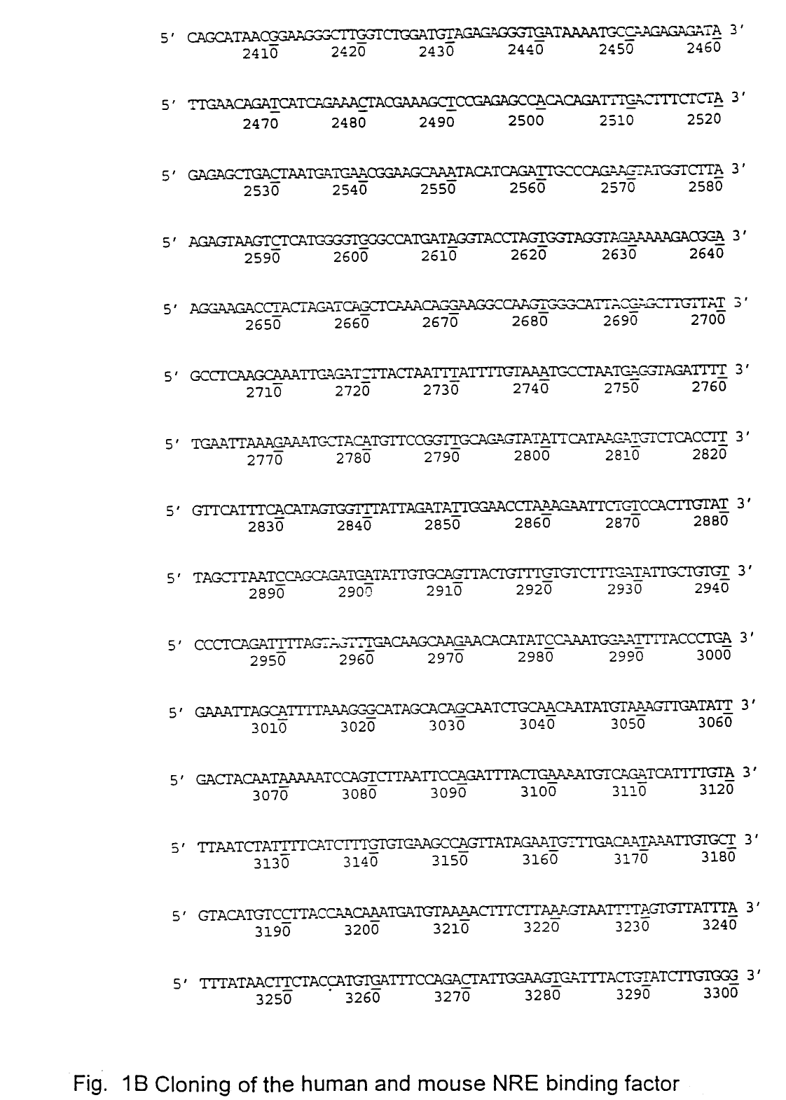

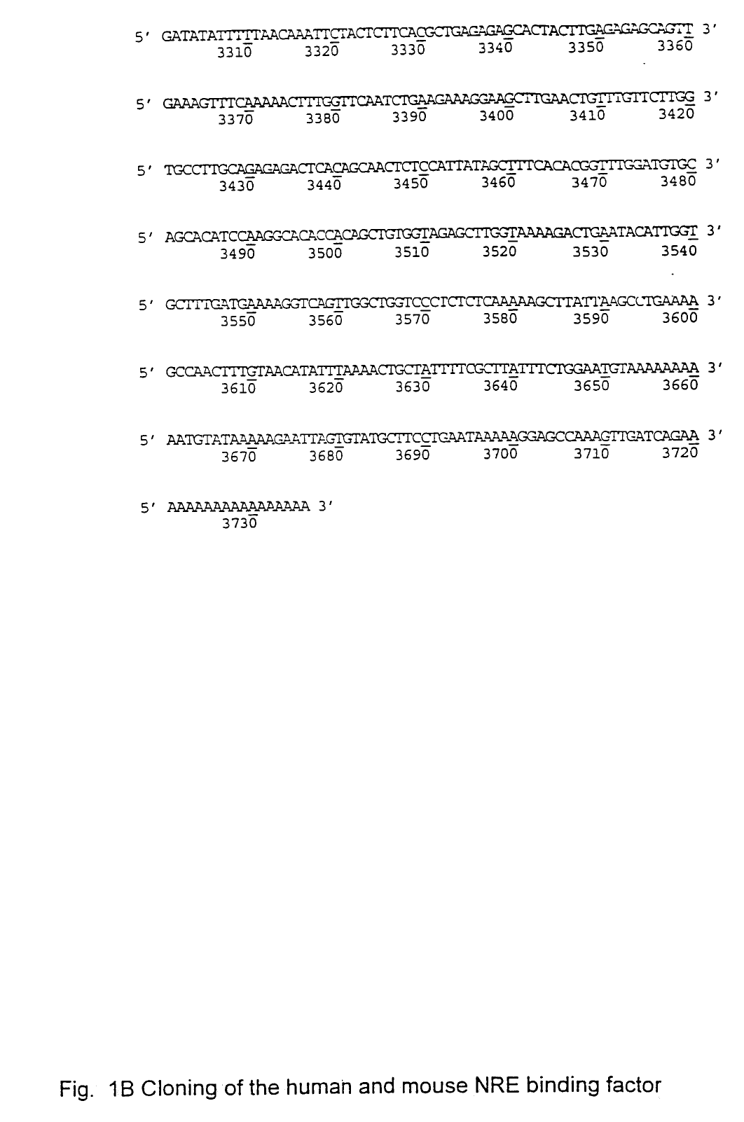

(a) having the sequence according to FIG. 1B or

(b) having the sequence according to FIG. 1B wherein

(i) at positions 984 to 1077 and

(ii) at positions 1897 to 1979

the nucleotides shown in FIG. 1B are replaced by those shown below them in the second row or

(c1) having the same number of nucleotides as the ssDNA according to (a) or

(c2) having a reduced number of nucleotides compared with the ssDNA according to (a)

wherein the ssDNA according to (c1) and (c2) is hybridizable with that according to (a) and/or (b).

A ssDNA according to the invention may comprise or have the nucleotide region

(i) of from position 654 to position 1817 or

(ii) of from position 1518 to 1817 (DNA binding domain=DBD) or

(iii) of from position 654 to position 1526 (silencer domain) or

(IV) of from position 1 to position 653 according to FIG. 1B or ssDNA

(v-i) having the same or a reduced number of nucleotides compared with the ssDNA according to (i) or

(v-ii) having the same or a reduced number of nucleotides compared with the ssDNA according to (ii) or

(v-iii) having the same or a reduced number of nucleotides compared with the ssDNA according to (iii) or

(v-iv) having the same or a reduced number of nucleotides compared with the ssDNA according to (iv)

wherein the ssDNA according (v-i), (v-ii), (v-iii) and (v-iv) is hybridizable with that according to (i), (ii), (iii) and (iv)

As regards a ssDNA according to (iv) or (v-iv) or a dsDNA or a RNA corresponding thereto, the following background is given. NRF mRNA has an extraordinarily long 5′untranslated region of 654 nucleotides containing several open reading frames. In principle, mRNAs which contain unusually long leader sequences with multiple upstream reading frames are good candidates for initiating translation via cap-independent internal ribosome binding mechanism (Sachs et al., 1997). The cap-independent internal initiation model was initially proposed in picornaviral mRNAs. These internal ribosome entry sites (IRES) have been successfully removed from their viral setting and linked to unrelated genes to produce polycistronic RNAs. A few cellular mRNAs have also been found to contain IRESs. As described so far, cellular IRESs display low efficiency in directing translation by internaL initiation. The strength of translation initiation from IRESs is equal or weaker when compared to cap-dependent translation initiation.

Another embodiment of the invention concerns a ssDNA which is characterized in that it is complementary to a ssDNA as described before.

Hybridization condition for hybridizable ssDNAs (c1), (c2) or (iv) as defined before are, for example, at a temperature of at least 25° C. and a 1 M sodium chloride concentration.

According to another embodiment the invention concerns the dsDNA consisting of a ssDNA as described above and its complementary strand.

According to another embodiment the invention concerns a RNA.

(a) having a sequence corresponding to that of a DNA according to the invention as described above or

(b) having a sequence corresponding to that of a RNA according to (a) but in anti-sense or

(c) being a degradation product o a RNA according to (a) or (b) being degraded in a manner known per se.

According to another embodiment the invention concerns a vector comprising a dsDNA as described. The vector may comprise

(i) a dsDNA consisting of a ssDNA as described in paragraphs (i), (ii) and (iii) before and a complementary strand or

(ii) a ssDNA according to paragraphs (i), (ii) and (iii) as described before and a complementary strand coding the same amino acid as a dsDNA according to (i) but comprised by the vector in antisense direction.

A vector according to the invention may be used for the transformation of cells and organisms for transient or for permanent expression of a protein encoded by the dsDNA comprised by the vector.

Another embodiment of the invention concerns a protein encoded by a ssDNA or a dsDNA as described before, optionally fused with another functional protein or one or more functional fragments thereof. As regards these functional fragments, they may be fused with the protein according to the invention as interspiced fragments. Of course, the protein according to the invention may be an unfused protein.

Another embodiment of the invention concerns a protein (dominant negative mutant) which can be obtained by

(a) mutating the nucleotide sequence of a ssDNA according to the invention in a manner known per se,

(b) expressing the mutated ssDNA (ssDNAs) in a manner known per se

(c) subjecting the expression product(s) to a competing test for inhibition of transcription with a protein encoded by the unmodified ssDNA (starting ssDNA) and

(d) isolating a protein which acts as a dominant negative mutant of the protein encoded by the unmodified ssDNA.

A protein which represses the human IFN-β promoter was postulated earlier. However, the properties of NRF and the effects exerted upon overexpression of its sense and antisense RNA are unexpected because

crosslinking analysis revealed not one but at least two proteins of about 100 kDa molecular mass and

NRF has no homology to teh DSP-1 protein which was thought to be the Drosophila homologue to the IFN-β repressor (17).

The protein(s) according to the invention encoded by the human gene has (have) the following properties:

It binds specifically DNA-sequences which are identical or related to the NRE-motif. The NREs are contained in a number of promoters of human genes.

It affects transcription of a number of cellular and viral genes, e. g. it represses the background activity of NF-_{K}B/rel-binding enhancer elements. By these properties, it constitutively represses the activity of a number of cellular genes.

Inactivation of endogenous NRE expression leads to the induction of cellular genes, i. g. the IFN-β gene.

NRF can be regarded as a modulator protein of NF-_{K}B family members controlling genes of significant biomedical importance such as those encoding inflammatory cytokines, MHC proteins, cell adhesion molecules, and viruses. Based on this it represents a molecular target in the development of novel anti-inflammatory therapies for a variety of pathologic disorders such as ischemia, hemorrhagic and septic shock, allograft rejection, bacterial meningitis, acute airway inflammation and the pulmonary complications induced by cardiopulmonary bypass. Furthermore, this might apply for certain cancers and other diseases.

Another embodiment of the invention concerns a use of

(i) a ssDNA according to the invention or

(ii) a dsDNA according to the invention or

(iii) a vector according to the invention or

(iv) a protein according to the invention

(a) for identifying and developing agonists and antagonists of NRF-functions,

(b) for the development of improved antisense NRF and ribozymes,

(c) for the detection and diagnosis of transient or permanent regulatory disorders of NF_{K}B/rel- and/or NRF-regulated physiological patterns in animals and humans or

(d) for therapy development and treatment of diseases, especially rheumatoid, arthritis, inflammations, infectious diseases, tumors and/or genetic diseases, or

(e) for gene therapy in animals and humans.

Finally, another embodiment of the invention concerns a use of a RNA according to the invention having a sequence according to that of a DNA according to (iv) oder (v-iv) as indicated above.

Said use can be as IHRES element in a polycistronic expression vector for application in an eucaryotic cell or a transgenic animal.

Finally, said use can be as translational enhancer in a monocistronic or a polycistronic expression vector for application in an eucaryotic cell or a transgenic animal.

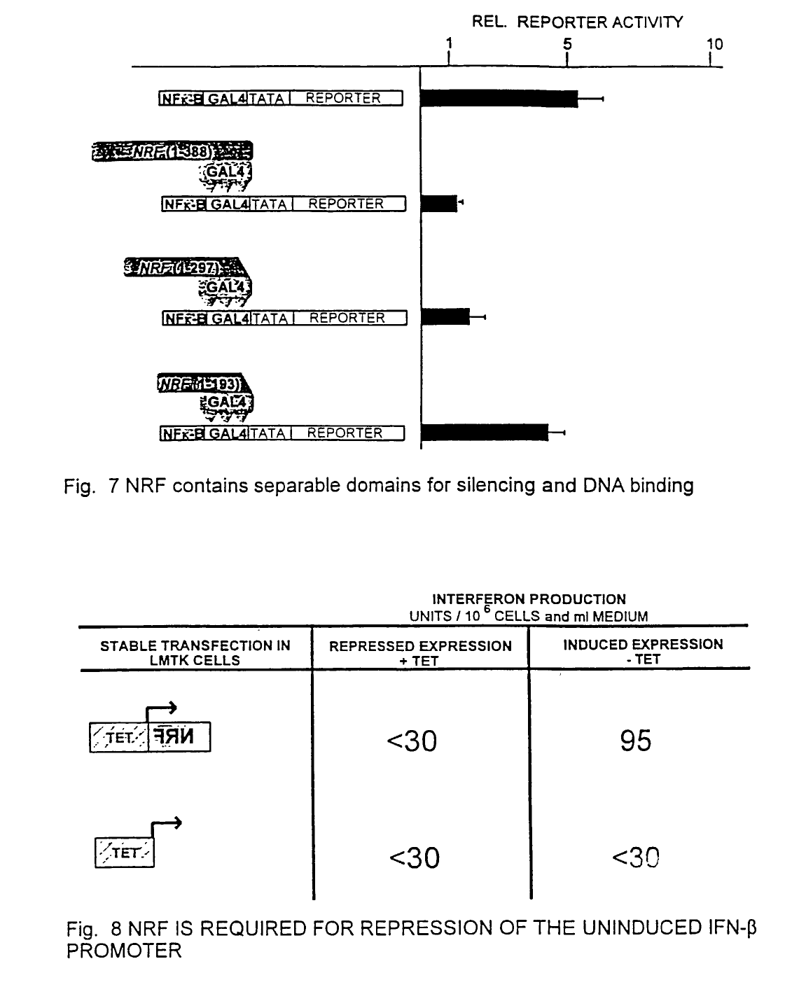

The following FIGS. 1 to 9 and the following examples explain the invention in greater detail.

### EXAMPLE 1

1. Cloning of the Human and Mouse NRE Binding Factor (Negative Regulatory Factor):

A) The method relies on the expression of human cDNA inserts from HeLa cells in bacteriophage lambda gtll. Fusion protein adsorbed onto nitrocellulose filters (NC) is probed with radioactive, double-stranded NRE-sequence as a ligand; NRE cf. Nourbakhsh et al. in EMBO J., 12 (1993) 451-459. Specific NRE-binding signals were detected on filters and corresponding bacteriophage plaques were isolated. Specific DNA-binding signals were detected on duplicate filters probed either with NRE-sequence or mutant NRE-sequence.

A cDNA clone coding for a 44 kd protein was detected with high specificity for NRE-sequence. Bacterial cells were infected with distinct number of the recombinant bacteriophage carrying cDNA of 44 kd protein and duplicate filters were probed with labeled DNA as indicated. The filters were exposed to X-ray film overnight, generating autoradiographic images (FIG. 1A).

B) The human NRF cDNA was used as hybridization probe to screen for homologous sequences in a cDNA-bank from mouse embryos (day 11) at reduced stringency. Identified clones were isolated, characterized and sequenced. FIG. 1B shows the sequence alignment of human (middle line) and murine (underneath) NRF cDNA. The protein coding region of human NRF cDNA is given by indicated amino acid sequence above the human cDNA sequence.

2. Structure of the Human Negative Regulatory Factor (NRF)

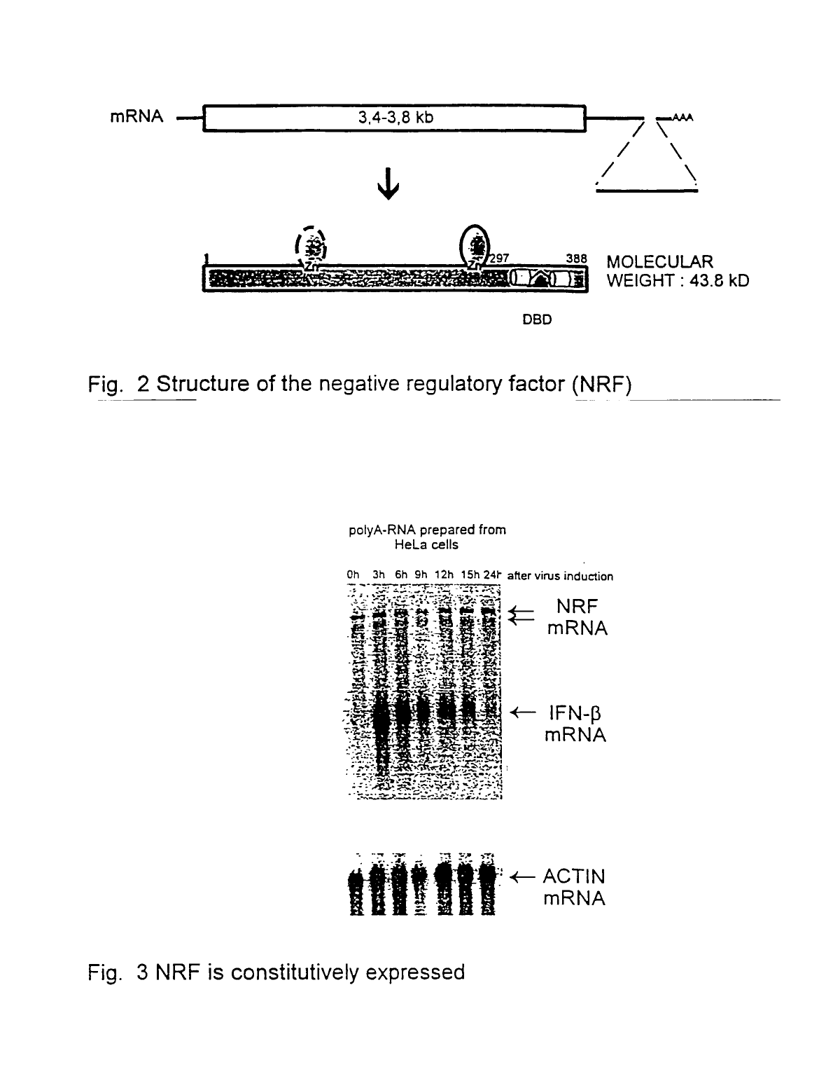

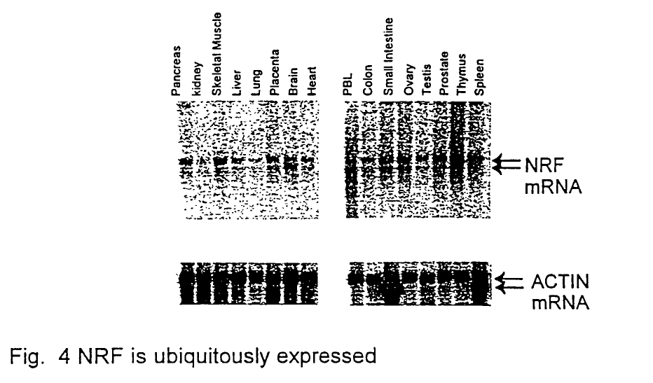

Two mRNAs with different 3′untranslated regions were identified, coding for NRF in human cells. The size of the NRF mRNAs is 2.8 and 3.8 kb (FIGS. 3 and 4). The coding region of mRNA is indicated by an open bar in FIG. 2. In the same figure, the protein sequence of NRF consisting of 388 aa is demonstrated by a dark bar. The silencer domain of NRF consisting of first 291 aa contains two different zinc-fingers indicated by bubbles. The DNA binding domain of NRF (DBD) is within 100 amino acids of the C-terminal end and contains a helix-loop-helix motif (FIG. 2).

3. NRF is Constitutively Expressed:

The expression level of NRF was determined by Northern blot analysis using poly(A)-RNA from HeLa cells. In FIG. 3, two different mRNA corresponding to NRF are indicated (top). Both mRNAs are constitutively expressed and their level of expression is not altered after treatment of the cells with Newcastle disease virus. Interferon-β mRNA (middle) shows a typical induction of the cells. Actin mRNA indicates equal amounts of RNA on each lane (obtained by rehybridization).

4. NRF is Ubiquitously Expressed:

The expression level of NRF in different human tissues was determined by Northern blot analysis. Two different mRNA corresponding to NRF are indicated on the top. Actin mRNA is indicated showing an equal amount of RNA on each lane (FIG. 4).

5. NRF Binds to NRE Regulated Promoters in vivo:

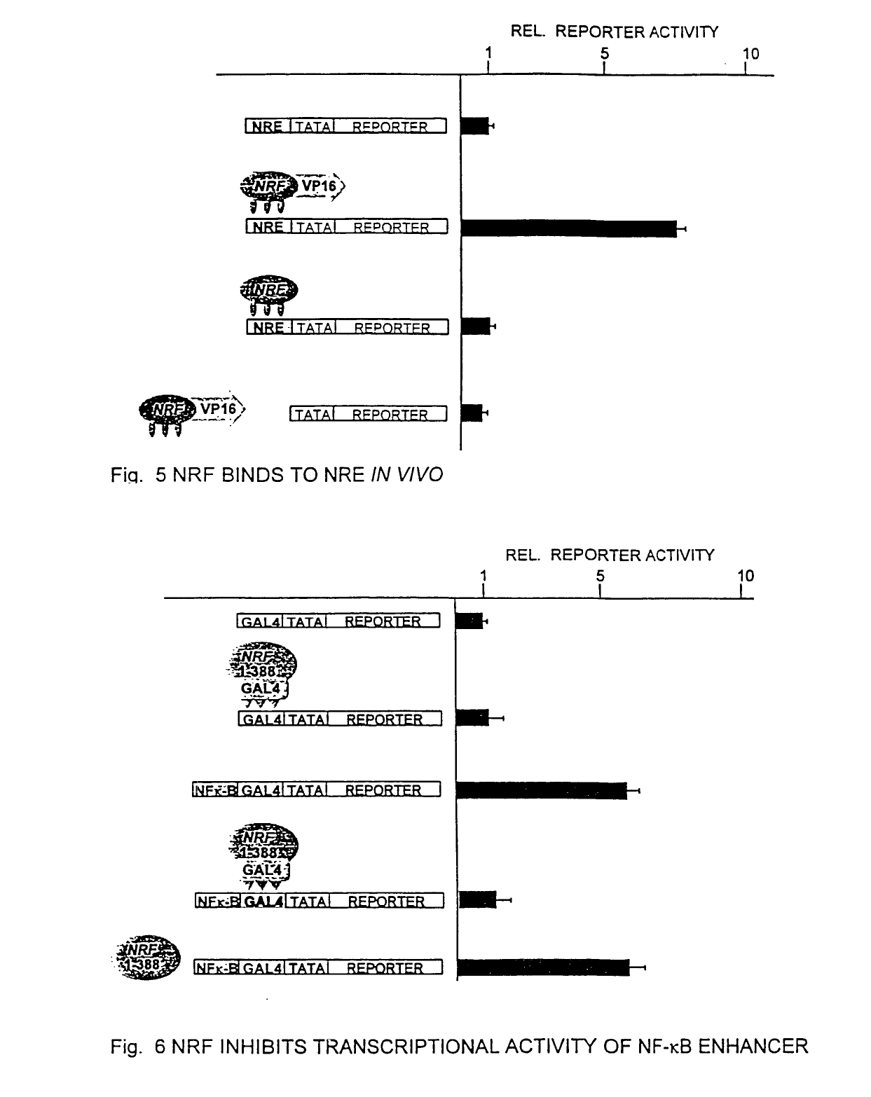

DNA-binding activity of NRF in vivo was tested using constructs encoding either NRF or a chimaeric protein consisting of full length NRF and the VP16 activating domain from Herpes simplex virus. Expression of these chimaeric proteins were carried out in murine cells habouring various reporter constructs as indicated by bars on the left side of FIG. 5. The relative reporter activities are indicated by black bars on the right side (FIG. 5). The data show that while w.t. NRF does not alter the expression of the NRE-promoter, the synthetic fusion protein NRF-VP16 acts as a transcriptional activator of this construct. The repressor activity of NRF is not detectable in this assay since the reporter does not contain an NF-_{K}B/rel site. The fusion protein does not affect promoters which do not contain NRE-recognition sites.

6. NRF Inhibits Transcriptional Activity of NF-_{K}B Promoters:

Transcriptional activity of NRF was tested using constructs encoding either NRF or a chimaeric protein consisting of NRF and the GAL4 DNA-binding domain. Expression of these chimaeric proteins was carried out in murine cells harbouring the indicated reporter constructs. These contain GAL4 and NF-_{K}B binding sites as indicated. The relative reporter activities are given as black bars on the right side (FIG. 6).

The fusion protein GAL4-NRF exhibits its repressor activity combined with the DNA-binding property of GAL4. The promoter in which an NF-_{K}B site enhances the basal promoter activity is inhibited by the fusion protein expression, whereas the basal promoter is not affected. W.t. NRF does not affect the reporter gene expression since its promoter does not contain an NRE.

7. NRF Contains Separable Domains for Silencing and DNA-binding.

The 100 C-terminal amino acids of NRF are sufficient to bind to NRE. This was demonstrated by expression in E. coli of an incomplete cDNA clone. The experiment was as outlined in FIG. 1A.

In order to define the repressor domain of NRF constructs were designed encoding chimaeric proteins containing C-terminal deletions of NRF and the GAL4 DNA-binding domain. These chimaeric proteins were expressed in murine cells harbouring a reporter construct containing GAL4 and NF-_{K}B binding sites as indicated in FIG. 8. The relative reporter activities are indicated by black bars.

8. Antisense Expression of NRF Affects Endogenous Gene Expression:

A conditioned expression plasmid encoding human NRF antisense cDNA (bp 1-344, aa 1-114) was stably transferred into murine C243 cells. IFN activity in the supernatant was measured only upon induction of NRF antisense expression. This result indicates that endogenous NRF expression which represses a constitutive activity of the IFN-β promoter is eliminated by the antisense RNA.

### EXAMPLE 2

An oligonucleotide having the sequence of FIG. 1B can also be obtained by screening material of a public gene library by means of a synthetic primer, having a subsequence according to FIG. 1B, in a manner non per se and by isolating the oligonucleotide wanted.

Example

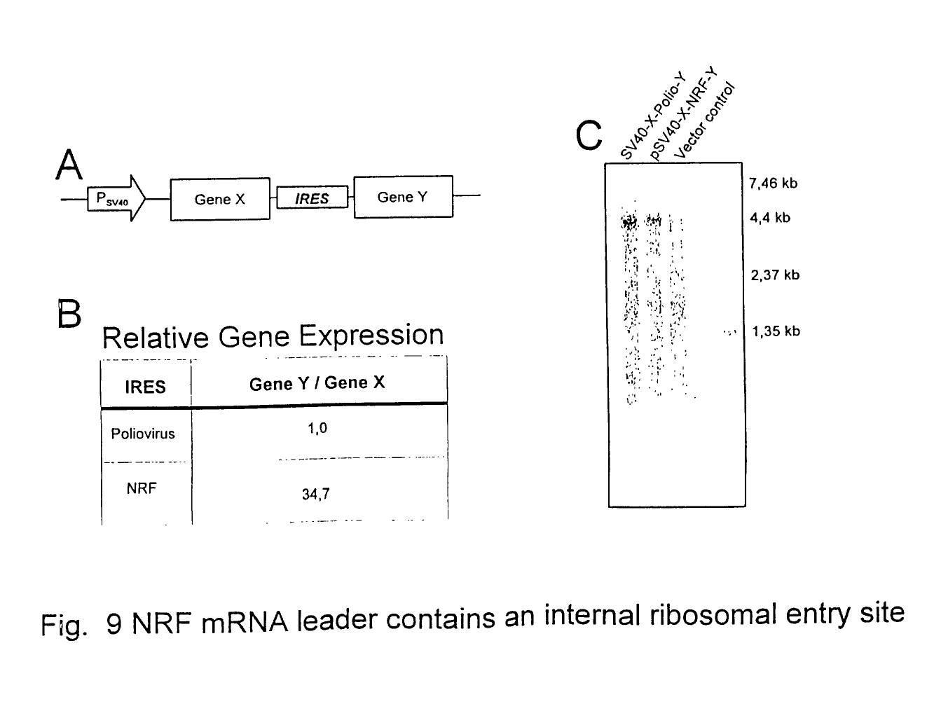

IRES activity of human NRF 5′UTR was determined by dicistronic reporter plasmids in vivo. These were constructed by using the Renilla luciferase gene and the firefly luciferase gene under the transcriptional control of the SV40 promoter. The 5′UTR of NRF or Poliovirus was inserted between two cistrons of this plasmid (FIG. 9A). Resulting constructs were transiently expressed in murine C243 cells. This was done to test NRF 5′UTR for IRES activity and secondly, to compare the efficiency of human NRF 5′UTR with Poliovirus IRES. The expression levels of the reporter genes were compared and indicated as relative gene expression. As shown in FIG. 9B, the efficiency of NRF IRES is 34,7 fold higher than the efficiency of the Poliovirus IRES. We performed Northern blot analysis to provide a control for the equal transcription of bicistronic mRNAs and to exclude transcript starts within IRES sequences. As shown in FIG. 9, the size and expression level of both mRNAs is indistinguishable. Thus, translation initiation takes place from the human NRF 5′UTR sequence element. Furthermore, the strength of this IRES element is higher than that of all other known IRES elements. Since the strength of the Polio virus IRES is about ⅓ of the cap dependent translation initiation (Dirks et al., 1993), the strength of the NRF IRES element is higher than that of cáp-dependent translation.

### REFERENCES

Sachs;A., B., Sarnow; P., Hentze; M., W., 1997, Starting at the Beginning, Middle, and End: Translation Initiation in Eukaryotes, Cell 89, 831-838

Dirks, W., Wirth, M. and Hauser, H., 1993: Bicistronic transcription units for gene expression in mammalian cells. Gene 128, 247-249( )

### REFERENCES

1. Beg, A. A., W. C. Sha, R. T. Bronson, S. Ghosh, and D. Baltimore. 1995. Embryonic lethality and liver degeneration in mice lacking the ReIA component of NF-kappaB. Cell. 376:165-170

2. Beraud, C., S.-C. Sun, P. Ganchi, D. W. Ballard, and W. C. Greene. 1994. Human T-cell leukemia virus type I Tax associates with and is negatively regulated by the NF-kappaB2 p100 gene product: Implications for viral latency. Mol. Cell. Biol.. 14:1374-1382

3. Boehnlein, E., J. W. Lowenthal, M. Siekevitz, D. W. Ballard, R. Franza, and W. C. Green. 1988. The same indiucible nuclear protein regulates mitogen activation of both the interleukin-2 receptor-alpha gene and type 1 HIV. Cell. 53:827-836

4. Colbère-Garapin, F., F. Horodniceanu, P. Khourilsky, and A. C. Garapin. 1981. A new domonant hybrid selective marker for higher eukaryotic cells. J. Mol. Biol.. 150:1-13

5. Dignam, J. D., R. M. Lebovitz, and R. G. Roeder. 1983. Accurate transcription initiation by RNA polymerase II in a soluble extract from isolated mammalian nuclei. Nucleic Acids Res.. 11:1475-1489

6. Drew, P. D., G. Franzoso, L. M. Carlson, W. E. Biddison, U. Siebenlist, and K. Ozato. 1995. Interferon regulatory factor-2 physically interacts with NF-kappaB in vitro and inhibits NF-kappaB induction of major histocompatibility class I and Beta2-microglobulin gene expression in transfected human neuroblastoma cells. J. Neuroimmunol.. 63:157-162

7. Du, H., A. L. Roy, and R. G. Roeder. 1993. Human transcription factor USF stimulates transcription through the initiator elements ofthe HIV-1 and the Ad-ML promoters. EMBO J.. 12:501-511

8. Fried, M., and D. M. Crothers. 1981. Equilibria and kinetics of lac repressor-operator interactions by polyacrylamide gel elecrophoresis. Nucleic Acids Res.. 9:6505-6525

9. Fujita, T., Y. Kimura, M. Miyamoto, E. L. Barsoumian, and T. Taniguchi. 1989. Induction of endogenous IFN-α and IFN-β genes by a regulatory transcription factor, IRF-1. Nature (London). 337:270-272

10. Goodbourn, S., H. Burstein, and T. Maniatis. 1986. The human beta-interferon gene enhancer is under negative control. Cell. 45:601-610

11. Goodbourn, S., and T. Maniatis. 1988. Overlapping positive and negative regulatory domains of the human β-interferon gene. Proc. Natl. Acad. Sci. USA. 85:1447-1451

12. Herschbach, B. M., and A. D. Johnson. 1993. Transcriptional repression in eukaryotes. Annu. Rev. Cell Biol.. 9:479-509

13. Hirsch, M.-R., L. Gaugler, H. Deagostini-Bazin, and L. Bally-Cuif. 1990. Identification of Positive and Negative Regulatory Elements Governing Cell-Type-Specific Expression of the Neural Cell Adhesion Molecule Gene. Mol. Cell. Biol.. 10:1959-1968

14. Hiscott, J., D. Alper, L. Cohen, J. F. Leblanc, L. Sportza, A. Wong, and S. Xanthoudakis. 1989. Induction of human interferon gene expression is associated with a nuclear factor that interacts with the NF-kappaB site of the human immundeficiency virus enhancer. J. Virology. 63:2557-2566

15. John, S., R. B. Reeves, J.-X. Lin, R. Child, J. M. Leiden, C. B. Thompson, and W. J. Leonard. 1995. Regulation of cell-type-specific interleukin-2 receptor Alpha-chain gene expression: Potential role of physical interactions between Elf-1, HMG-I(Y), and NF-kappaB family proteins. Mol. Cell. Biol.. 15:1786-1796

16. Kanno, T., K. Brown, G. Franzoso, and U. Siebenlist. 1994. Kinetic analysis of human T-cell leukemia virus type I tax-mediated activation of NF-kappaB. Mol. Cell. Biol.. 14:6443-6451

17. Lehming, N., D. Thanos, J. M. Brickman, J. Ma, T. Maniatis, and M. Ptashne. 1994. An HMG-like protein that can switch a transcriptional activator to a repressor. Nature (London). 371:175-179

18. Lenardo, M. J., C.-M. Fan, T. Maniatis, and D. Baltimore. 1989. The involvement of NF-kappaB in β-interferon gene regulation reveals its role as widely inducible mediator of signal transduction. Cell. 57:287-294

19. Lindholm, P. F., M. Tamami, J. Makowski, and J. N. Brady. 1996. Human T-cell Iymphotropic virus type 1 Tax1 activation of NF-kappaB: Involvement of the protein kinase C pathway. J. Virol.. 70:2525-2532

20. Liou, H.-C., and D. Baltimore. 1993. Regulation of the NF-kappaB/rel transcription factor and IkappaB inhibitor system. Curr. Opin. Cell Biol.. 5:477-487

21. Liu, X., R. Ge, and R. P. Ricciardi. 1996. Evidence for the involvement of a nuclear NF-kappa inhibitor in global down-regulation of the major histocompatibility complex class I enhancer in adenovirus type 12-transformed cells. Mol. Cell. Biol.. 16:398-404

22. Lu, Y., N. Touzjian, M. Stenzel, T. Dorfman, J. G. Sodroski, and W. A. Haseltine. 1990. Identification of cis-acting repressive sequences within the negative regulatory element of human immunodeficiency virus type 1. J. Virol.. 64:5226-5229

23. Mielke., E., M. Nourbakhsh and H. Hauser. A new class of transcriptional repressor sequences which silence the activity of NF-κB/rel binding sites. In preparation

24. Munoz, E., and A. Israel. 1995. Activation of NFkappaB by Tax Protein of HTLV-1. immunology. 193:128-136

25. Nourbakhsh, M., K. Hoffmann, and H. Hauser. 1993. Interferon-β promoters contain a DNA element that acts as a position-independent silencer on the NF-kappaB site. EMBO J.. 12:451-459

26. Pazin, M. J., Sheridan, P. L., Cannon, K., Cao, Z. D., Keck, J. G., Kadonaga, J. T., Jones, and K. A.. 1996. NF-kappaB-mediated chromatin reconfiguration and transcriptional activation of the HIV-1 enhancer in vitro. Genes Dev.. 10:37-49

27. Reil, H., H. Kollmus, U. H. Weidle, and H. Hauser. 1993. A heptanucleotide sequence mediates ribosomal frameshifting in mammalian cells. J. Virol.. 67:5579-5584

28. Saksela, K., and D. Baltimore. 1993. Negative regulation of immunoglobulin kappa light-chain gene transcription by a short sequence homologous to the murine B1 repetitive element. Mol. Cell. Biol.. 13:3698-3705

29. Salvetti, A., A. Lilienbaum, Z. Li, D. Paulin, and L. Gazzolo. 1993. Identification of a negative element in the human vimentin promoter: modulation by the human T-cell leukemia virus type I Tax protein. Mol. Cell. Biol.. 13:89-97

30. Sambrook, J., E. F. Frisch, and T. Maniatis. 1989. Molecular Cloning: A Labatory Manual. Cold Spring Harbor Laboratory, Cold Spring Harbor (N.Y.).

31. Seiki, M., Y. Hattori, Y. Hirayama, and M. Yoshida. 1983. Human adult T-cell leukemia virus: complete nucleotide sequence of the provirus genome integrated in leukemia cell DNA. Proc. Natl. Acad. Sci. USA. 80:3618-3622

32. Siekevitz, M., S. F. Josephs, M. Dukovich, N. Peffer, F. Wong-Staal, and W. C. Greene. 1987. Activation of the HIV-1 LTR by T cell mitogens and the trans-activator protein of HTLV-I. Science. 238: 1575-1578

33. Sif, S., and T. Gilmore. 1994. Interaction of the v-Rell Oncoprotein with cellular Transcription Factor SP1. J. Virol.. 68:7131-7138

34. Smith, M. R., and W. C. Greene. 1989. The same 50-kDa cellular protein binds to the negative regulatory elements of the interleukin 2 receptor α-chain gene and the human immunodeficiency virus typ 1 long terminal repeat. Proc. Natl. Acad. Sci. USA. 86:8526-8530

35. Tanimura, A., H. Teshima, J. Fujisawa, and M. Yoshida. 1993. A new regulatory element that augments the Tax-dependent enhancer of human T-cell leukemia virus type 1 and cloning of cDNAs encoding its binding proteins. J. Virol.. 67:5375-5382

36. Thanos, D., and T. Maniatis. 1992. The high mobility group protein HMG I (Y) is required for NF-kappaB-dependent virus induction of the human IFN-Beta gene. Cell. 71:777-789

37. Thanos, D., and T. Maniatis. 1995. Virus induction of human IFNBeta gene expression requires the assembly of an enhanceosome. Cell. 83:1091-1100

38. Tillotson, L. G., T. C. Wang, and S. J. Brand. 1994. Activation of Gastrin Transcription in pancreatic insulinoma cells by a CACC promoter element and a 70-kDa sequence-specific DNA-binding protein. J. Biol. Chem.. 269:2234-2240

39. Visvanathan, K. V., and S. Goodbourn. 1989. Double-stranded RNA activates binding of NF-kappaB to an inducible element in the human beta-interferon promoter. EMBO J.. 8:1129-1138

40. Wang, T. C., and S. J. Brand. 1990. Islet Cell-specific Regulatory Domain in the Gastrin Promoter Contains Adjacent Positive and negative DNA Elements. J. Biol. Chem.. 265:8908-8914

41. Whelan, J., P. Ghersa, R. H. van Huijsduijnen, J. Gray, G. Chandra, F. Talabot, and J. F. Delamater. 1991. An NFkB-like factor is essential but not sufficient for cytokine induction of endothelial leukocyte adhesion molecule 1 (ELAM-1) gene transcription. Nucleic Acids Res.. 19:2645-2653

42. Wildemann, A. G., P. Sasson-Corsi, T. Grundström, M. Zenke, and P. Chambon. 1984. Stimulation of in vitro transcription from the SV 40 early promoter by the enhancer involves a specific trans- acting factor. EMBO J.. 3:3129-3133

What is claimed is:

1. An isolated single stranded polynucleotide comprising a polynucleotide sequence selected from the group consisting of SEQ ID NO: 1, SEQ ID NO: 3, SEQ ID NO: 5, SEQ ID NO: 7, SEQ ID NO: 9, SEQ ID NO: 11, fragments thereof, and polynucleotides complementary thereto.

2. An isolated single stranded polynucleotide comprising a polynucleotide sequence set forth in SEQ ID NO: 1 wherein the single stranded polynucleotide is hybridizable to a polynucleotide comprising a polynucleotide sequence selected from the group consisting of SEQ ID NO: 5, SEQ ID NO: 7, SEQ ID NO: 9, and SEQ ID NO: 11, at a temperature of at least 25° C. and a 1M sodium chloride concentration.

3. An isolated single stranded polynucleotide comprising a polynucleotide sequence set forth in SEQ ID NO: 3 wherein the single stranded polynucleotide is hybridizable to a polynucleotide comprising a polynucleotide sequence selected from the group consisting of SEQ ID NO: 5, SEQ ID NO: 7, SEQ ID NO: 9, and SEQ ID NO: 11, at a temperature of at least 25° C. and a 1M sodium chloride concentration.

4. A double stranded polynucleotide comprising a single stranded polynucleotide according to claim 1 and its complementary strand.

5. A double stranded polynucleotide comprising a single stranded polynucleotide according to claim 2 and its complementary strand.

6. A double stranded polynucleotide comprising a single stranded polynucleotide according to claim 3 and its complementary strand.

7. An RNA (a) comprising a sequence complementary to a polynucleotide according to claim 1, (b) according to (a) where the RNA is anti-sense RNA, or (c) being a degradation product of a RNA according to (a) or (b).

8. An RNA (a) comprising a sequence complementary to a polynucleotide according to claim 2, (b) according to (a) where the RNA is anti-sense RNA, or (c) being a degradation product of a RNA according to (a) or (b).

9. An RNA (a) comprising a sequence complementary to a polynucleotide according to claim 3, (b) according to (a) where the RNA is anti-sense RNA, or (c) being a degradation product of a RNA according to (a) or (b).

10. An RNA (a) comprising a sequence complementary to a polynucleotide according to claim 4, (b) according to (a) where the RNA is anti-sense RNA, or (c) being a degradation product of a RNA according to (a) or (b).

11. An RNA (a) comprising a sequence complementary to a polynucleotide according to claim 5, (b) according to (a) where the RNA is anti-sense RNA, or (c) being a degradation product of a RNA according to (a) or (b).

12. An RNA (a) comprising a sequence complementary to a polynucleotide according to claim 6, (b) according to (a) where the RNA is anti-sense RNA, or (c) being a degradation product of a RNA according to (a) or (b).

13. A vector which comprises a double stranded polynucleotide according to claim 4.

14. The vector according to claim 13 wherein the double stranded polynucleotide encodes a polypeptide in antisense direction.

15. A vector which comprises a double stranded polynucleotide according to claim 5.

16. The vector according to claim 15 wherein the double stranded polynucleotide encodes a polypeptide in antisense direction.

17. A vector which comprises a double stranded polynucleotide according to claim 6.

18. The vector according to claim 17 wherein the double stranded polynucleotide encodes a polypeptide in antisense direction.

19. A recombinant host cell comprising a polynucleotide selected from the group consisting of:
(a) a polynucleotide according to claim 1;
(b) a polynucleotide comprising a polynucleotide sequence encoding the polypeptide as set forth in SEQ ID NO: 2;
(c) a polynucleotide comprising a polynucleotide sequence which hybridizes to the complement of either of (a) or (b); and
(d) a double stranded polynucleotide comprising a single stranded polynucleotide according to (a), (b) or (c) and its complementary strand.

20. A method for producing an NRF polypeptide comprising the steps of:
(i) culturing a host cell according to claim 19 in growth medium under conditions suitable for expression of the NRF polypeptide; and
(ii) isolating the expressed NRF polypeptide from the cell or the medium.

21. The NRF polypeptide of claim 20, fragments thereof, and variants thereof.

22. An NRF polypeptide selected from the group consisting of SEQ ID NO: 2. SEQ ID NO: 4, SEQ ID NO: 6, SEQ ID NO: 8, SEQ ID NO: 10, fragments thereof, and variants thereof.

23. The polypeptide according to claim 22 further comprising another functional polypeptide or functional fragment thereof fused thereto.

24. The polypeptide according to claim 22 wherein the polypeptide is an unfused polypeptide.

25. A method of screening for a dominant negative mutant NRF polypeptide comprising:
(i) mutating the polynucleotide sequence of a single stranded polynucleotide according to claim 1 to a make a mutated single stranded polynucleotide;
(ii) expressing the mutated single stranded polynucleotide to produce an expression product;
(iii) subjecting the expression product to a competing test for inhibition of transcription with a polypeptide encoded by an unmodified single stranded polynucleotide; and
(iv) identifying as a dominant negative mutant NRF polypeptide an expression product that competed with the polypeptide encoded by the unmodified single stranded polynucleotide in the competing test of (c).

26. A dominant negative mutant protein identified in claim 25.

27. A method of identifying an antagonist of NRF polypeptide binding to a polynucleotide comprising the steps of:
(i) exposing an NRF polypeptide according to claim 22 to a polynucleotide under conditions which permit binding of NRF polypeptide to a polynucleotide in the presence and absence of a test compound;
(ii) measuring the binding of NRF polypeptide to a polynucleotide in the presence and absence of the test compound; and
(iii) identifying as antagonist a test compound by its ability to prevent binding of NRF polypeptide to a polynucleotide.

28. A method of identifying an agonist of NRF polypeptide binding to a polynucleotide comprising the steps of:
(i) exposing an NRF polypeptide according to claim 22 to a polynucleotide encoding a polypeptide under conditions which permit binding of NRF polypeptide to a polynucleotide in the presence and absence of a test compound;
(ii) measuring the polypeptide produced by binding of the test compound to the polynucleotide; and
(iii) identifying as agonist a test compound by its ability to further reduce polypeptide production in the presence as opposed to the absence of the test compound.

29. A method of preventing NRF polypeptide expression comprising introducing a polynucleotide according to claim 1 to a eucaryotic cell or a transgenic animal, wherein the polynucleotide encodes an NRF polypeptide in antisense.

30. A method of preventing NRF polypeptide expression comprising introducing an RNA (a) comprising a sequence complementary to a polynucleotide according to claim 1, (b) according to (a) where the RNA is antisense RNA, or (c) being a degradation product of an RNA according to (a) or (b) to a eucaryotic cell or a transgenic animal.

31. A ribozyme comprising an RNA (a) comprising a sequence complementary to a polynucleotide according to claim 1, (b) according to (a) where the RNA is antisense RNA, or (c) being a degradation product of an RNA according to (a) or (b).

32. A method of detecting and diagnosing transient or permanent regulatory disorders of NF_{K}B/rel- and/or NRF-regulated physiological patterns in animals comprising:
(i) determining the presence or amount of expression of an NRF polypeptide according to claim 22 in a biological sample; and
(ii) diagnosing transient or permanent regulatory disorders of NF_{K}B/rel- and/or NRF-regulated physiological patterns based on the presence or amount of expression of the NRF polypeptide.

33. A method of treating or ameliorating a disease selected from the group consisting of rheumatoid arthritis, inflammations, infectious diseases, tumors, and genetic diseases, the method comprising administering a polynucleotide of claim 1 in an effective amount.

34. A method of treating or ameliorating a disease selected from the group consisting of rheumatoid arthritis, inflammations, infectious diseases, tumors, and genetic diseases. the method comprising administering a polynucleotide of claim 4 in an effective amount.

35. A method of treating or ameliorating a disease selected from the group consisting of rheumatoid arthritis, inflammations, infectious diseases, tumors, and genetic diseases. the method comprising administering a polypeptide of claim 22 in an effective amount.

36. A method of gene therapy in animals comprising administering to an animal a polynucleotide of claim 1.

37. A method of gene therapy in animals comprising administering to an animal a polynucleotide of claim 4.

38. An RNA comprising a sequence complementary to a polynucleotide (a) comprising a polynucleotide sequence set forth in SEQ ID No. 11, (b) according to (a) where the RNA is anti-sense RNA, or (c) being a degradation product of an RNA according to (a) or (b).

39. A method of expressing an open reading frame in a eucaryotic cell or a transgenic animal comprising:
(i) inserting an RNA according to claim 38 as an internal ribosome entry site element in a polycistronic expression vector containing two or more open reading frames; and
(ii) introducing the polycistronic expression vector to the eucaryotic cell or the transgenic animal.

40. A polycistronic expression vector which comprises two or more open reading frames and an RNA according to claim 38, wherein the RNA serves as an internal ribosome entry site.

41. A method of enhancing translation in an eucaryotic cell or a transgenic animal comprising:
(i) inserting an RNA according to claim 38 as a translational enhancer in a monocistronic expression vector or a polycistronic expression vector; and
(ii) introducing the monocistronic expression vector or polycistronic expression vector to the eucaryotic cell or the transgenic animal.

42. A monocistronic expression vector or a polycistronic expression vector which comprises one or more open reading frames and an RNA according to claim 38, wherein the RNA serves as a translational enhancer.


SEQUENCE LISTING (11 sequences)

SEQ ID NO: 1 - DNA, 3736 bp, Homo sapiens
  CDS (654)..(1817)
cagagtaatgacatggttccttccatcctccaaaggtgaccaataatagtttgtaagtatcattatgaactaatgaattttcaacatatttgatatatttcaatccattgccatcattgttcttatcgatatttgagttggctcactttgccagtaagagtctattcaaattggcttctgagtccatttgacacaacacctttgatctttgacagtttccttggttttaggtgctagatgatttctcaggctcaccttagacatttcctgccacagacttagaatcagccatttctctaaggaccctgattccatttcatgagaaatgatagagaccacaatcaaaacaagtcatgaatttatactgatattttcaattcaaattaaagatgaggtttttgctaaatttttttgagtttatatttgtatgtcttatgctgaaaaatcttgtttcctaattagtaacataattattcatttgatgggtaaatattttagggccgattctttggttttatagccaagataccctgttgataaagtcttgtgggagcaattataagactggcttattttgaagctttttaaaaaagacatccttacctgttttaactgtagattatattaacttaaataggtacagcccacgcttgatggaaaaaattctccaaatggctgaaggtattgatattggggagatgccttcatatgatctggtgctgtccaaaccttccaaaggtcaaaaacgccacctctcaacatgtgatggtcaaaatcctcctaaaaagcaagccggttccaaattccatgcgagacctcgttttgagcctgtacattttgtagctagtagttcaaaagatgaaggacaggaagatccttatggccctcaaacaaaagaggtaaatgaacaaacacattttgccagcatgccaagagacatctaccaagattatactcaagactctttcagtatacaagatgggaattctcagtattgtgattcatcaggattcattctcacaaaagaccagcctgtaacagccaacatgtattttgacagtgggaaccctgccccaagcaccacatcacagcaggcaaactctcagtcaactcctgagccttcaccatcacagacatttcccgagtctgtggtagccgagaagcagtattttattgaaaaattaacggcgacaatctggaagaacctttctaatccagaaatgacttctggatctgataaaattaattatacatatatgttaactcgttgtattcaggcgtgtaagacaaatcctgagtatatatatgctcctttaaaggaaattcctcctgccgacatccccaaaaataaaaaacttctaactgatggctatgcttgtgaagttagatgccaaaatatctacttaactacaggttatgctggcagcaagaatgggtccagggatcgagctacagagctagctgtaaaactcttgcagaaacgtattgaagttagagttgtccggcggaaattcaagcatacatttggagaggacctcgtggtgtgtcagattggcatgtcctcctatgaatttcctccagctctgaagccaccagaagacctggtggtgctgggtaaagatgcttccgggcagccaatttttaatgcttctgccaaacactggaccaattttgtcattacagaaaatgcaaatgatgcaattggtatccttaacaattctgcctcattcaacaagatgtcaattgaatacaaatatgagatgatgccaaatcgcacatggcgttcgtcgagtgtttttacaagatcactgcttagctgaaggttatggaaccaagaaaacaagtaaacatgcagctgccgacgagtttgaaaattcttcaaaaacacagcccacttatccatctgtcaaaagttcacaatgccatacaggctcttcacccagaggatctggaaagaagaaagatataaaggctctgtagtttatgagaattcttcaaatcccgtgtgcacgctgaacgacacagctcagtttaaccgaatgacagttgagtatgtctatgaaaggatgacaggcctccgctggaaatgcaaagtgattctagagagtgaagtaattgcagaagcagttggggtgaagaaaactgtcaaatataagctgctggggaagctgtgaaaaccctcaaaaagacccagcaactgtcattaacaacttgaagaaaggagctgttgaagatgtgatttcaagaaatgaaattcagggccgctcagcagaggaggcttacaaacagcaaatcaaagaagataatattggaaatcagctgctgagaaagatgggttggactggtggtggtttaggtaaatctggtgagggcatacgggagcctatctcagtgaaagagcagcataacggaagggcttggtctggatgtagagagggtgataaaatgccaagagagatattgaacagatcatcagaaactacgaaagctccgagagccacacagatttgactttctctagagagctgactaatgatgaacggaagcaaatacatcagattgcccagaagtatggtcttaagagtaagtctcatggggtgggccatgataggtacctagtggtaggtagaaaaagacggaaggaagacctactagatcagctcaaacaggaaggccaagtgggcattacgagcttgttatgcctcaagcaaattgagatcttactaatttattttgtaaatgcctaatgaggtagatttttgaattaaagaaatgctacatgttccggttgcagagtatattcataagatgtctcaccttgttcatttcacatagtggtttattagatattggaacctaaagaattctgtccacttgtattagcttaatccagcagatgatattgtgcagttactgtttgtgtctttgatattgctgtgtccctcagattttagtagtttgacaagcaagaacacatatccaaatggaattttaccctgagaaattagcattttaaagggcatagcacagcaatctgcaacaatatgtaaagttgatattgactacaataaaaatccagtcttaattccagatttactgaaaatgtcagatcattttgtattaatctattttcatctttgtgtgaagccagttatagaatgtttgacaataaattgtgctgtacatgtccttaccaacaaatgatgtaaaactttcttaaagtaattttagtgttatttatttataacttctaccatgtgatttccagactattggaagtgatttactgtatcttgtggggatatatttttaacaaattctactcttcacgctgagagagcactacttgagagagcagttgaaagtttcaaaaactttggttcaatctgaagaaaggaagcttgaactgtttgttcttggtgccttgcagagagactcacagcaactctccattatagctttcacacggtttggatgtgcagcacatccaaggcacaccacagctgtggtagagcttggtaaaagactgaatacattggtgctttgatgaaaaggtcagttggctggtccctctctcaaaaagcttattaagcctgaaaagccaactttgtaacatatttaaaactgctattttcgcttatttctggaatgtaaaaaaaaaatgtataaaaagaattagtgtatgcttcctgaataaaaaggagccaaagttgatcagaaaaaaaaaaaaaaaaaa

SEQ ID NO: 2 - protein, 388 aa, Homo sapiens
MEKILQMAEGIDIGEMPSYDLVLSKPSKGQKRHLSTCDGQNPPKKQAGSKFHARPRFEPVHFVASSSKDEGQEDPYGPQTKEVNEQTHFASMPRDIYQDYTQDSFSIQDGNSQYCDSSGFILTKDQPVTANMYFDSGNPAPSTTSQQANSQSTPEPSPSQTFPESVVAEKQYFIEKLTATIWKNLSNPEMTSGSDKINYTYMLTRCIQACKTNPEYIYAPLKEIPPADIPKNKKLLTDGYACEVRCQNIYLTTGYAGSKNGSRDRATELAVKLLQKRIEVRVVRRKFKHTFGEDLVVCQIGMSSYEFPPALKPPEDLVVLGKDASGQPIFNASAKHWTNFVITENANDAIGILNNSASFNKMSIEYKYEMMPNRTWRSSSVFTRSLLS

SEQ ID NO: 3 - DNA, 3736 bp, Mus musculus
  CDS (654)..(1817)
  misc_feature (1008): N = A or C or G or T
cagagtaatgacatggttccttccatcctccaaaggtgaccaataatagtttgtaagtatcattatgaactaatgaattttcaacatatttgatatatttcaatccattgccatcattgttcttatcgatatttgagttggctcactttgccagtaagagtctattcaaattggcttctgagtccatttgacacaacacctttgatctttgacagtttccttggttttaggtgctagatgatttctcaggctcaccttagacatttcctgccacagacttagaatcagccatttctctaaggaccctgattccatttcatgagaaatgatagagaccacaatcaaaacaagtcatgaatttatactgatattttcaattcaaattaaagatgaggtttttgctaaatttttttgagtttatatttgtatgtcttatgctgaaaaatcttgtttcctaattagtaacataattattcatttgatgggtaaatattttagggccgattctttggttttatagccaagataccctgttgataaagtcttgtgggagcaattataagactggcttattttgaagctttttaaaaaagacatccttacctgttttaactgtagattatattaacttaaataggtacagcccacgcttgatggaaaaaattctccaaatggctgaaggtattgatattggggagatgccttcatatgatctggtgctgtccaaaccttccaaaggtcaaaaacgccacctctcaacatgtgatggtcaaaatcctcctaaaaagcaagccggttccaaattccatgcgagacctcgttttgagcctgtacattttgtagctagtagttcaaaagatgaaggacaggaagatccttatggccctcaaacaaaagaggtaaatgaacaaacacattttgccagcatgccaagagacatctaccaagattatactcaagactctttcagtatacaagatgggaattctcaatattgtaattcatcangatttattttcacaaaagaccagcctgtancaaccaacatgtattttgacagtgggaancctgncccaagcaccacatcacagcaggcaaactctcagtcaactcctgagccttcaccatcacagacatttcccgagtctgtggtagccgagaagcagtattttattgaaaaattaacggcgacaatctggaagaacctttctaatccagaaatgacttctggatctgataaaattaattatacatatatgttaactcgttgtattcaggcgtgtaagacaaatcctgagtatatatatgctcctttaaaggaaattcctcctgccgacatccccaaaaataaaaaacttctaactgatggctatgcttgtgaagttagatgccaaaatatctacttaactacaggttatgctggcagcaagaatgggtccagggatcgagctacagagctagctgtaaaactcttgcagaaacgtattgaagttagagttgtccggcggaaattcaagcatacatttggagaggacctcgtggtgtgtcagattggcatgtcctcctatgaatttcctccagctctgaagccaccagaagacctggtggtgctgggtaaagatgcttccgggcagccaatttttaatgcttctgccaaacactggaccaattttgtcattacagaaaatgcaaatgatgcaattggtatccttaacaattctgcctcattcaacaagatgtcaattgaatacaaatatgagatgatgccaaatcgcacatggcgttcgtcgagtgtttttacaagatcactgcttagctgaaggttatggaaccaagaaaacaagtaaacatgcagctgccgacgagtttgaaaattcttcaaaaacacagcccacttatccatctgtcaaaagttcccagtgtcactcaggcttttcacccaaaggatccggaaagaagaaagatatcaaagatcttgtgtttatgagaattcttcaaatcccgtgtgcacgctgaacgacacagctcagtttaaccgaatgacagttgagtatgtctatgaaaggatgacaggcctccgctggaaatgcaaagtgattctagagagtgaagtaattgcagaagcagttggggtgaagaaaactgtcaaatataagctgctggggaagctgtgaaaaccctcaaaaagacccagcaactgtcattaacaacttgaagaaaggagctgttgaagatgtgatttcaagaaatgaaattcagggccgctcagcagaggaggcttacaaacagcaaatcaaagaagataatattggaaatcagctgctgagaaagatgggttggactggtggtggtttaggtaaatctggtgagggcatacgggagcctatctcagtgaaagagcagcataacggaagggcttggtctggatgtagagagggtgataaaatgccaagagagatattgaacagatcatcagaaactacgaaagctccgagagccacacagatttgactttctctagagagctgactaatgatgaacggaagcaaatacatcagattgcccagaagtatggtcttaagagtaagtctcatggggtgggccatgataggtacctagtggtaggtagaaaaagacggaaggaagacctactagatcagctcaaacaggaaggccaagtgggcattacgagcttgttatgcctcaagcaaattgagatcttactaatttattttgtaaatgcctaatgaggtagatttttgaattaaagaaatgctacatgttccggttgcagagtatattcataagatgtctcaccttgttcatttcacatagtggtttattagatattggaacctaaagaattctgtccacttgtattagcttaatccagcagatgatattgtgcagttactgtttgtgtctttgatattgctgtgtccctcagattttagtagtttgacaagcaagaacacatatccaaatggaattttaccctgagaaattagcattttaaagggcatagcacagcaatctgcaacaatatgtaaagttgatattgactacaataaaaatccagtcttaattccagatttactgaaaatgtcagatcattttgtattaatctattttcatctttgtgtgaagccagttatagaatgtttgacaataaattgtgctgtacatgtccttaccaacaaatgatgtaaaactttcttaaagtaattttagtgttatttatttataacttctaccatgtgatttccagactattggaagtgatttactgtatcttgtggggatatatttttaacaaattctactcttcacgctgagagagcactacttgagagagcagttgaaagtttcaaaaactttggttcaatctgaagaaaggaagcttgaactgtttgttcttggtgccttgcagagagactcacagcaactctccattatagctttcacacggtttggatgtgcagcacatccaaggcacaccacagctgtggtagagcttggtaaaagactgaatacattggtgctttgatgaaaaggtcagttggctggtccctctctcaaaaagcttattaagcctgaaaagccaactttgtaacatatttaaaactgctattttcgcttatttctggaatgtaaaaaaaaaatgtataaaaagaattagtgtatgcttcctgaataaaaaggagccaaagttgatcagaaaaaaaaaaaaaaaaaa

SEQ ID NO: 4 - protein, 388 aa, Mus musculus
  misc_feature 119: Xaa = any or unknown amino acid
MEKILQMAEGIDIGEMPSYDLVLSKPSKGQKRHLSTCDGQNPPKKQAGSKFHARPRFEPVHFVASSSKDEGQEDPYGPQTKEVNEQTHFASMPRDIYQDYTQDSFSIQDGNSQYCNSSXFIFTKDQPVXTNMYFDSGXPXPSTTSQQANSQSTPEPSPSQTFPESVVAEKQYFIEKLTATIWKNLSNPEMTSGSDKINYTYMLTRCIQACKTNPEYIYAPLKEIPPADIPKNKKLLTDGYACEVRCQNIYLTTGYAGSKNGSRDRATELAVKLLQKRIEVRVVRRKFKHTFGEDLVVCQIGMSSYEFPPALKPPEDLVVLGKDASGQPIFNASAKHWTNFVITENANDAIGILNNSASFNKMSIEYKYEMMPNRTWRSSSVFTRSLLS

SEQ ID NO: 5 - DNA, 1164 bp, Homo sapiens
  CDS (1)..(1164)
atggaaaaaattctccaaatggctgaaggtattgatattggggagatgccttcatatgatctggtgctgtccaaaccttccaaaggtcaaaaacgccacctctcaacatgtgatggtcaaaatcctcctaaaaagcaagccggttccaaattccatgcgagacctcgttttgagcctgtacattttgtagctagtagttcaaaagatgaaggacaggaagatccttatggccctcaaacaaaagaggtaaatgaacaaacacattttgccagcatgccaagagacatctaccaagattatactcaagactctttcagtatacaagatgggaattctcagtattgtgattcatcaggattcattctcacaaaagaccagcctgtaacagccaacatgtattttgacagtgggaaccctgccccaagcaccacatcacagcaggcaaactctcagtcaactcctgagccttcaccatcacagacatttcccgagtctgtggtagccgagaagcagtattttattgaaaaattaacggcgacaatctggaagaacctttctaatccagaaatgacttctggatctgataaaattaattatacatatatgttaactcgttgtattcaggcgtgtaagacaaatcctgagtatatatatgctcctttaaaggaaattcctcctgccgacatccccaaaaataaaaaacttctaactgatggctatgcttgtgaagttagatgccaaaatatctacttaactacaggttatgctggcagcaagaatgggtccagggatcgagctacagagctagctgtaaaactcttgcagaaacgtattgaagttagagttgtccggcggaaattcaagcatacatttggagaggacctcgtggtgtgtcagattggcatgtcctcctatgaatttcctccagctctgaagccaccagaagacctggtggtgctgggtaaagatgcttccgggcagccaatttttaatgcttctgccaaacactggaccaattttgtcattacagaaaatgcaaatgatgcaattggtatccttaacaattctgcctcattcaacaagatgtcaattgaatacaaatatgagatgatgccaaatcgcacatggcgttcgtcgagtgtttttacaagatcactgcttagc

SEQ ID NO: 6 - protein, 388 aa, Homo sapiens; identical to SEQ ID NO: 2

SEQ ID NO: 7 - DNA, 300 bp, Homo sapiens
  CDS (1)..(300)
catacatttggagaggacctcgtggtgtgtcagattggcatgtcctcctatgaatttcctccagctctgaagccaccagaagacctggtggtgctgggtaaagatgcttccgggcagccaatttttaatgcttctgccaaacactggaccaattttgtcattacagaaaatgcaaatgatgcaattggtatccttaacaattctgcctcattcaacaagatgtcaattgaatacaaatatgagatgatgccaaatcgcacatggcgttcgtcgagtgtttttacaagatcactgcttagc

SEQ ID NO: 8 - protein, 100 aa, Homo sapiens
HTFGEDLVVCQIGMSSYEFPPALKPPEDLVVLGKDASGQPIFNASAKHWTNFVITENANDAIGILNNSASFNKMSIEYKYEMMPNRTWRSSSVFTRSLLS

SEQ ID NO: 9 - DNA, 873 bp, Homo sapiens
  CDS (1)..(873)
atggaaaaaattctccaaatggctgaaggtattgatattggggagatgccttcatatgatctggtgctgtccaaaccttccaaaggtcaaaaacgccacctctcaacatgtgatggtcaaaatcctcctaaaaagcaagccggttccaaattccatgcgagacctcgttttgagcctgtacattttgtagctagtagttcaaaagatgaaggacaggaagatccttatggccctcaaacaaaagaggtaaatgaacaaacacattttgccagcatgccaagagacatctaccaagattatactcaagactctttcagtatacaagatgggaattctcagtattgtgattcatcaggattcattctcacaaaagaccagcctgtaacagccaacatgtattttgacagtgggaaccctgccccaagcaccacatcacagcaggcaaactctcagtcaactcctgagccttcaccatcacagacatttcccgagtctgtggtagccgagaagcagtattttattgaaaaattaacggcgacaatctggaagaacctttctaatccagaaatgacttctggatctgataaaattaattatacatatatgttaactcgttgtattcaggcgtgtaagacaaatcctgagtatatatatgctcctttaaaggaaattcctcctgccgacatccccaaaaataaaaaacttctaactgatggctatgcttgtgaagttagatgccaaaatatctacttaactacaggttatgctggcagcaagaatgggtccagggatcgagctacagagctagctgtaaaactcttgcagaaacgtattgaagttagagttgtccggcggaaattcaagcatacattt

SEQ ID NO: 10 - protein, 291 aa, Homo sapiens
MEKILQMAEGIDIGEMPSYDLVLSKPSKGQKRHLSTCDGQNPPKKQAGSKFHARPRFEPVHFVASSSKDEGQEDPYGPQTKEVNEQTHFASMPRDIYQDYTQDSFSIQDGNSQYCDSSGFILTKDQPVTANMYFDSGNPAPSTTSQQANSQSTPEPSPSQTFPESVVAEKQYFIEKLTATIWKNLSNPEMTSGSDKINYTYMLTRCIQACKTNPEYIYAPLKEIPPADIPKNKKLLTDGYACEVRCQNIYLTTGYAGSKNGSRDRATELAVKLLQKRIEVRVVRRKFKHTF

SEQ ID NO: 11 - DNA, 653 bp, Homo sapiens
cagagtaatgacatggttccttccatcctccaaaggtgaccaataatagtttgtaagtatcattatgaactaatgaattttcaacatatttgatatatttcaatccattgccatcattgttcttatcgatatttgagttggctcactttgccagtaagagtctattcaaattggcttctgagtccatttgacacaacacctttgatctttgacagtttccttggttttaggtgctagatgatttctcaggctcaccttagacatttcctgccacagacttagaatcagccatttctctaaggaccctgattccatttcatgagaaatgatagagaccacaatcaaaacaagtcatgaatttatactgatattttcaattcaaattaaagatgaggtttttgctaaatttttttgagtttatatttgtatgtcttatgctgaaaaatcttgtttcctaattagtaacataattattcatttgatgggtaaatattttagggccgattctttggttttatagccaagataccctgttgataaagtcttgtgggagcaattataagactggcttattttgaagctttttaaaaaagacatccttacctgttttaactgtagattatattaacttaaataggtacagcccacgcttg